(19) United States
(12) Patent Application Publication
(10) Pub. No.: US 2003/0206957 A1
(43) Pub. Date: Nov. 6, 2003

(54) Orally administered medicament delivery systems

(76) Inventor: George H. Scherr, Park Forest, IL (US)

Correspondence Address: George H. Scherr, Ph.D., 33 Monee Road, Park Forest, IL, 60466, US

(21) Appl. No.: 10/138,118

(22) Filed: May 6, 2002

(51) Int. Cl. A61K 38/38; A61K 38/43; A61K 31/715; A61K 31/56; A61K 39/00; A61K 31/375; A61K 9/14

(52) U.S. Cl. 424/488; 514/54; 424/184.1; 424/94.1; 514/171; 514/12; 514/474

(57) ABSTRACT

The specification discloses an alginate composition in which medicaments or cells may be interspersed with aqueous insoluble alginate molecules, so that pellets prepared by this procedure would survive the stomach contents, any enzymatic activity contained therein, as well as the low pH, and gradually dissolve in the intestinal tract behaving as a controlled release system of any specific medicament or cells including, but not limited to, vaccines that are entrapped in the alginate coacervate. Orally administered particles so prepared could be used to eliminate the need for parenteral needle inoculation of various medicaments in man and animals.

### DESCRIPTION OF THE PRIOR ART

Oshlack, et al., in their U.S. Pat. No. 6,261,599, describe the preparation of extrudates, which include hydrophobic chemicals in which therapeutic agents are mixed, such as various narcotic drugs and other agents, and then the final material is extruded through a fine opening in the form of filaments. The filaments are then divided into unit doses, either by measuring the length, inserting them into capsules, or their being coated in the form of a compressed tablet.

It should be noted that the essential tenets of Oshlack, et al.'s patent involve hydrophobic materials in which the carrier material constitute such products as mineral or vegetable oils as well as natural and synthetic waxes. The hydrophobic materials, with which the drugs are mixed, are compounds such as acrylic and methacrylic polymers including hydrogenated castor oil or hydrogenated vegetable oils.

It is clearly the purpose of the Oshlack patent to permit a gradual disintegration and/or dilution of this hydrophobic mixture, which mixture will slowly begin to dissolve when ingested and exposed to gastric as well as intestinal fluids. Therefore, the medicaments incorporated into such a hydrophobic composition could not be protein in nature or products that would be degraded by any of the proteolytic enzymes present in the stomach nor could they be susceptible to being inactivated by the relatively low pH present in the stomach.

In the patent by German, et al., U.S. Pat. No. 6,258,789, the inventors describe a procedure in which DNA is directed to specific cells of the small and large intestines for the purpose of introducing a therapeutic gene that actually transforms the cells of the intestine to a desired genetic capability to produce compounds of medicinal value. The workers point out that, although it is known that any DNA that enters the gastro-intestinal tract would be destroyed by the digestive process, either by stomach acid or intestinal enzymes, the oral delivery of DNA, the subject of the invention by German, et al. is successful in overcoming these attributes and survives the stomach acid or intestinal enzymes. The so-called “gene pill” (capsule) of this invention can be protected against stomach acids or intestinal enzymes by being coated with various substances despite the fact that the preferred capsule contains naked DNA and is used without conventional formulations to provide survival in the stomach or intestinal tract. In most of the examples cited by German, et al. the transforming DNA is introduced into the intestinal lumen by a surgical procedure.

In the U.S. Pat. No. 6,265,389 by Burke, the inventor microencapsulates oligonucleotides in a polymer matrix. The oligonucleotide is first stabilized by complexing it with a polycationic complexing agent such as protamine, albumin, spennidine or spermine; suitable metal cations can also be used for complexing with the oligonucleotide. The stabilized oligonucleotide is then dissolved in a bio-compatible polymer in which the oligonucleotide particles would be non-reactive, such as methylene chloride, ethyl acetate, acetone, or other polar organic liquids. The dispersion of the stabilized oligoneucleotide and the polymer solution are then processed to create droplets, which are frozen to form micro droplets. The frozen micro droplets are then exposed to a liquid or solid non-solvent, such as ethanol or ethanol mixed with hexane. The solvent in the frozen micro droplets is then extracted as a solid and/or liquid into the non-solvent to form stabilized oligonucleotide-containing micro particles.

The micro particles of the oligonucleotide thus prepared can be injected either intravenously or subcutaneously to have their desired pharmacological effect.

In the U.S. Pat. No. 6,248,353 of Singh, the inventor prepares liposomes, which have membrane protein incorporated into the liposomes. The preformed liposomes are made by a method comprising combining a mixture of phospholipids with a solution of at least one type of unsaturated fatty acid. The release of any component contained within these liposomes would be discharged in their entirety into the tissues where they are injected or introduced in their entirety since the liposomes are acting as a protective surface of a sphere in which are contained the essential components whose activity are desired to be introduced into a viable entity.

Attributes of the Invention Described Herein

The primary necessary characteristic of the invention described herein is the ability of a medicament to survive the relatively harsh conditions of the stomach with regard to low pH and the presence of proteolytic enzymes. The second characteristic that is achieved by this invention requires that the method of immobilization of the medicament whose activity is desired for man or animal be gradually released from the particles in which they are entrapped thus avoiding a sudden total release of the product as frequently occurs with other systems of delivery such as the use of albumin or in various liposomes. An additional attribute that must be considered as a necessary one for the entrapment of medicaments that are to be swallowed dictates that the material utilized for the entrapment of a medicament be biologically acceptable and produce no antigenic or other toxic effect to the host that is so treated. It has been well documented in the profession that many of the gut-associated lymphoid tissues, such as Peyer's Patches can and do respond to an antigenic stimulus so that survival of a desired antigen past the stomach and its gradual release into the intestinal tract can have the effect of eliciting an immune response without the necessity of parenteral injection of such an antigen.

Although calcium alginate has been used as a mechanism of entrapment of various products, they primarily have been utilized for immobilizing cells or enzymes for the in vitro induction of a biologically required product. Cho and Einolf have reviewed in some detail the use of such immobilized systems in which various cells including microbial cells were immobilized and acted on substrates, which produced products of biological desirability (see “Application of Immobilized Cells and Enzymes for Pharmaceutical Production” by Michael Y. Cho and David M. Einolf, Pharmaceutical Manufacturing, October 1985, page 39). Calcium alginate has also been utilized by the wine industry in entrapping yeast cells, which were able to maintain their metabolic activity and convert sugars into alcohol so avoiding the sediment that takes place when yeast cells are not so entrapped (see “A Champagne Maker Finds a New Fizz,” in the Jan. 6, 1986, issue of Fortune, page 65).

The mixture of a soluble alginate with a desired medicament and its precipitation as an aqueous-insoluble alginate matrix in which the medicament is entrapped has advantages over prior art in that:

The alginates are on the GRAS list (Generally Recognized As Safe) and therefore are acceptable as providing a matrix for medicaments that are to be swallowed by man or animals.

The reaction between an aqueous soluble alginate in which a particular medicament has been dissolved or dispersed can result in the precipitation of aqueous insoluble alginate pellets of varying size, the preparation of which can take place over a wide range of pH. Consequently the pH of the reaction between the aqueous soluble alginate and the divalent or trivalent cation metal ion salt that will complex with the alginate to form an aqueous insoluble alginate gel, may be dictated by the optimal pH for the stability of the particular medicament that is mixed with or dispersed in the aqueous soluble alginate.

The reaction between an aqueous soluble alginate and a divalent or trivalent cation metal ion salt that would react with the water-soluble alginate to form an aqueous insoluble alginate gel, can take place over a relatively wide range of temperature; again dictated by any requirements of the particular medicament entrapped therein.

Since the stomach contents and the digestive environment of different animals and man may vary, then the degree of the hardness of the alginate matrix in which is entrapped a particular medicament can be altered, literally at will. This can be done by an appropriate choice of the particular alginate and/or the concentration of calcium or other di- or trivalent cation metal salt that will complex with the particular alginate to form aqueous insoluble gels. Thus the versatility of the invention described herein would permit the entrapment in an aqueous insoluble alginate gel of a very wide variety of medicaments that might require a wide range of optimum pH's.

Another salient advantage of the invention described herein permits the drying of the aqueous insoluble alginate pellets in which matrix a particular medicament has been entrapped so that the medicament so entrapped can be stored for extended periods of time prior to being administered by the oral route or possible mixed with food or drink to permit their ready swallowing and passage unchanged through the stomach.

It is generally known that sodium salts of organic acids, such as sodium citrate, sodium desoxycholate, sodium hexametaphosphate, and many others will readily dissolve a calcium alginate product. The rate of the solution of the aqueous insoluble alginate-medicament product can be controlled by altering the hardness of the aqueous insoluble alginate matrix into which the medicament has been entrapped by varying the concentration of the soluble calcium salt into which the alginate-medicament mixture has been submerged; and can also be controlled by the judicious choice of the specific aqueous soluble alginate, which is made insoluble by an appropriate di- or trivalent cation earth metal salt.

Should it be desirable to provide a medicament that may have a very high viscosity, such as a high concentration of albumin, it is possible to utilize an aqueous soluble alginate that has a very low viscosity at the appropriate concentrations required to precipitate the alginate-medicament mixture by having it submerged into a calcium or other metallic salt that would precipitate the coercive mixture. Thus, note the following very wide range of viscosities that are readily available that provide an additional advantage to the very broad choice of medicaments that could be utilized within the scope of the invention described herein.

| | | |
|---|---|---|
| Sodium Alginate | Concentration | Viscosity |

| | | |
|---|---|---|
| Kelco Keltone Hv | 2.0% aqueous solution | 4,500 cP |
| Kelco Kelgin Hv | 2.0% aqueous solution | 10,000 cP |
| Kelco Kelgin RL | 2.0% aqueous solution | 30 cP |

Another salient advantage of the invention described herein permits the utilization of a divalent or trivalent cation metal salt, which will complex the aqueous soluble alginate mixture with a medicament that might be desirable. Thus complexing the aqueous soluble alginate composition can take place with calcium salts and also other salts, which will precipitate the alginate molecule such as those of aluminum, zinc, iron (ferric or ferrous), copper, chromium, or silver as examples that may be readily utilized to precipitate the coercive alginate mixtures containing the appropriate medicament.

In general, calcium salts are usually utilized to precipitate alginates because of its ready availability and the well-known sensitivity of alginates to precipitation by calcium ions. It is generally known in the profession that sodium alginate in aqueous solution is highly sensitive to precipitation by calcium ions and that even as low a concentration of 1 to 100,000 of sodium alginate in aqueous solution, can result in precipitation of calcium alginate in the presence of solutions of calcium chloride. It is therefore another salient advantage of the invention described herein that relatively high concentrations of medicaments can be mixed with the aqueous alginate and precipitation can occur with relatively low amounts of calcium or other metal ions without impairing the performance of the medicaments whose activity is desired.

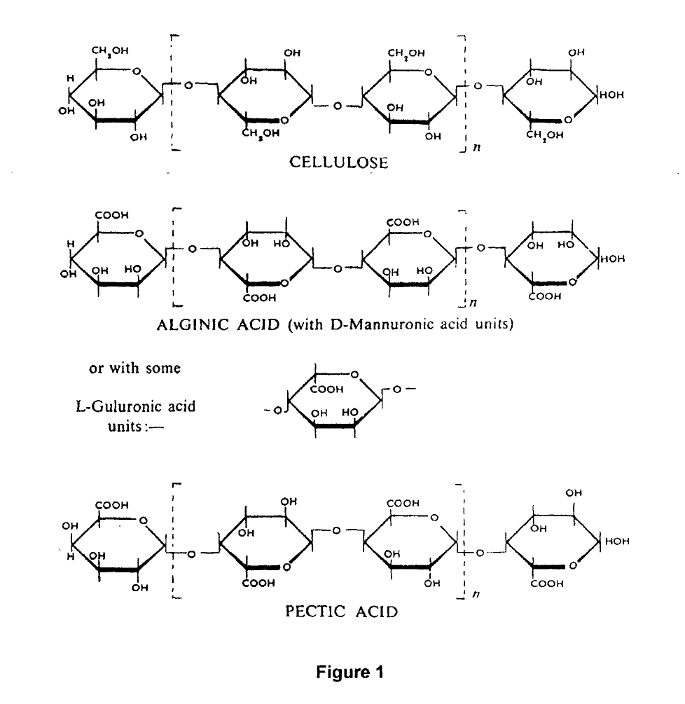

It has been observed that the very close molecular relationship between pectic acid and alginic acid (see FIG. 1) has made it feasible to precipitate pectins with calcium ions in the same manner as calcium ions can be used to precipitate sodium alginate to an aqueous insoluble calcium alginate state. The degree of esterification (DE) of the pectin in general should be less than 50% so that a lower concentration of calcium/or other polyvalent cations that precipitate the pectin may be utilized. Further, the degree of amidation (DA) is preferred to be 25% or less for more optimum reaction with calcium ions in forming gels of the pectin. Thus, should it be desirable to prepare the products, the subject of this invention, with pectins instead of alginates this would readily be feasible since pectins are also on the GRAS list and would be biologically acceptable as a matrix for the medicaments, whose activity it is desired to release into the intestinal tract.

Having set forth the tenets of the invention contained herein, the following non-limiting examples illustrate various compositions that are inherent in our invention.

### DESCRIPTION OF THE PREFERRED EMBODIMENTS

### EXAMPLE 1

Place 4000 ml of deionized water into a plastic container and, with stirring, slowly add 100 grams of Kelco HV brand of sodium alginate. The stirring should be sufficiently vigorous to form a vortex in the water so that the sodium alginate added to the water is directed into the middle of the vortex to ensure a very efficient dispersion of the alginate particles and so increase the rate of solution. The mixture is stirred until all of the sodium alginate has been dissolved.

To 1125 ml of the 2.5% sodium alginate solution prepared as above, add the following ingredients:

75 ml of glycerin,

6.9 ml of the surface active agent polyoxyethylene—polyoxypropylene block polymer, (L64, Wyandotte Corp.)

85 ml of the Dirofilaria immitis vaccine as prepared in the U.S. Pat. No. 4,761,281.

After stirring for approximately ten minutes to ensure that all of the ingredients have been dispersed, the incoming tube connected to a variable peristaltic pump is lowered into the container holding the ingredients as prepared above. In the event that the vaccine may contain sediment or any particulate suspensions that are desirable, the alginate vaccine mixture may be continuously stirred utilizing a magnetic stirrer.

To the output hose of the pump is inserted a number 26 hypodermic syringe needle, which is then suspended over a 3000 ml beaker containing 2000 ml of 5% calcium chloride. The peristaltic pump is started at a reduced rate such as 5 ml per minute and the drops coming out of the syringe needle will fall into the 5% calcium chloride immediately forming small gel pellets. The hose containing the exit needle for the mixture that has been prepared should be approximately 12 inches above level of the 5% calcium chloride so that the droplets coming out of the syringe needle will form into a sphere when they strike the surface of the calcium chloride.

It will be found that as the calcium ions gradually diffuse into the pellets so formed. The pellets will gradually begin to sink in the calcium chloride container until they reach the bottom, because the specific gravity of the finished pellet is greater than that of the 5% calcium chloride solution.

The calcium alginate-vaccine pellets so prepared can then be recovered with a small mesh aluminum container, washed with deionized distilled water and then layered onto a clean filter paper sheet to air dry overnight. The dried calcium alginate-vaccine pellets thus prepared can be stored in a closed container for at least one year without any detectable degradation of the activity of the vaccine.

### EXAMPLE 2

Twenty (20) grams of calcium-sensitive pectin such as pectins which have a degree of esterification of less than 30 percent, which may be procured from Sigma Chemical Company or from Hercules, Inc., is dissolved in 1500 ml of hot deionized water that has been heated to between 80°-90° C. To the 1500 ml of the pectin solution prepared as above, add the following ingredients:

75 ml of glycerin

6.9 ml of the surface active agent polyoxethylene-polyoxypropylene block polymer (L64, Wyandotte Corp.)

6.0 ml of the surface active agent polyoxyethylenesorbitan monooleate (Tween 80®, Atlas Chemical Industries, Inc.)

15 grams of sodium tetraborate dissolved in 50 ml of hot deionized water

To the above solution and after it is cooled to room temperature add 50 ml of the peptide adjuvant n-acetylmuramyl-L-alanyl-d-isoglutamine (Sigma) then add 200 μg ω-agatoxin TK (Sigma).

After stirring for approximately ten minutes to ensure that all of the ingredients have been dispersed, the incoming tube connected to a variable peristaltic pump is lowered into the container holding the ingredients as prepared above. In the event that the components may contain sediment or any particulate suspensions that are desirable, the pectin mixture may be continuously stirred utilizing a magnetic stirrer.

To the output hose of the pump is inserted a number 26 hypodermic syringe needle, which is then suspended over a 3000 ml beaker containing 2000 ml of 5% calcium chloride.

The pellets so prepared can then be recovered from the solution with a small aluminum mesh container, as described in Example 1 above, washed, and then air-dried or dried by lyophilization as may be dictated by the use of the pellets.

### EXAMPLE 3

Prepare 1125 ml of the 2.5% sodium alginate solution as prepared in Example 1 above.

Prepare a mixture of antibiotics by adding 230 ml of the zinc salt of bacitracin, having a concentration of 67 IU/mg, to 10 ml to of deionized water. Neomycin sulphate powder assaying as 704 mcg neomycin/mg of material is added to 10 ml of the deionized water in an amount of 135 mg. Polymyxin B sulphate containing 8547 units of polymyxin B/mg of powder is added to 10 ml of deionized water in an amount of 22.6 mg.

The three separate solutions are stirred until all of the antibiotics have been dissolved and then they are mixed, to form a total of 30 ml of solution.

The mixture of antibiotics so prepared is now added to the alginate solution as prepared above, following which, add:

75 ml of glycerin,

6.9 ml of the surface active agent polyoxyethylene—polyoxypropylene block polymer, (L64, Wyandotte Corp.)

After stirring for approximately ten minutes to ensure that all of the ingredients have been dispersed, the incoming tube connected to a variable peristaltic pump is lowered into the container holding the ingredients as prepared above. In the event that the vaccine may contain sediment or any particulate suspensions that are desirable, the alginate vaccine mixture may be continuously stirred utilizing a magnetic stirrer.

To the output hose of the peristaltic pump is inserted a number 26 hypodermic syringe needle, which is then suspended over a 3000 ml beaker containing 2000 ml of 5% calcium chloride. The peristaltic pump is started at a reduced rate such as 5 ml per minute and the drops coming out of the syringe needle will fall into the 5% calcium chloride immediately forming small gel pellets. The hose containing the exit needle for the mixture that has been prepared should be approximately 12 inches above level of the 5% calcium chloride so that the droplets coming out of the syringe needle will form into a sphere when they strike the surface of the calcium chloride.

The pellets can then be recovered as described in Example 1 above and then appropriately dried in air or lyophilization as required.

The above descriptions and examples illustrate particular constructions including the preferred embodiments of the solutions. However, the invention is not limited to the precise constructions described herein, but, rather, all modifications and improvements thereof encompassed within the scope of the invention.

The sodium alginate principally utilized in the examples described herein was one having an aqueous viscosity of 4500 cP at 2.0% concentration. It is clear that other sodium alginates having other viscosities may be utilized without deviating from the novelty of the revelations contained in this patent as long as the alginate is of a concentration and viscosity that can be reasonably dispensed into a solution of a calcium or other anion precipitating molecule to result in an aqueous insoluble alginate salt into which matrix a medicament is entrapped.

Although the alginate used in the examples described herein was sodium alginate, it is clear that other water soluble alginates may be utilized without deviating from the novelty of the invention described herein such as water soluble ammonium alginate, magnesium alginate, or potassium alginate.

It is well known in the profession that various glycols will act as plasticizers and may be used to improve the flexibility of alginate films or fibers. The plasticizer that we have principally used in the examples described herein has been glycerin because of its low cost and ready availability. It is clear however that other plasticizers may be utilized such as propylene glycol or ethylene glycol without deviating from the novelty of the invention described herein.

In the examples cited herein, calcium chloride has been utilized to provide the calcium ion which precipitates the insoluble calcium alginate, which serves to entrap into the calcium alginate matrix other components as described herein. It is clear, as has been mentioned, that other salts may be utilized to precipitate the alginate such as those of aluminum, zinc, copper, chromium, or silver and these insoluble alginates may readily be utilized to precipitate the coercive alginate mixtures described in the Examples provided herein without deviating from the essential merits of this invention. However, since the alginate compositions are to be utilized on biological tissues, the particular salt utilized to precipitate the alginate should be dictated by any restraints of toxicity or other untoward reactions that might result from their use.

Many of the examples described herein utilize the surface-active agents such as Pluronic L64. Surface-active agents are utilized primarily to effect a dispersion between the non-aqueous miscible components utilized in achieving a coercive mixture with the aqueous soluble sodium alginate in order to insure homogeneity throughout the solutions that are then precipitated as insoluble alginate compositions. Other surface-active agents, such as the Na salt of dodecyl SO₄ (sodium lauryl sulfate) or a member of the group of Tweens: (Tween 20, polyoxyethylene sorbitan monolaurate; Tween 40, polyoxyethylene sorbitan monopalmitate; or Tween 85, polyoxyethylene sorbitan trioleate) may be incorporated into the alginate composition without deviating from the novelty of the invention described herein.

Notice that the example number 1 herein, utilizes a 26 hypodermic syringe needle to dispense the mixture prepared in example 1. It is clear that hypodermic needles of varying sizes could be utilized depending upon the size of the pellet that may be desired without in any way deviating from the basic tenets of the invention described herein. In addition, although one needle is described to drop the solutions into the calcium chloride solution as described in example 1, it is readily feasible to use a multitude of hypodermic needles attached to a manifold so that six or seven, or eight drops can be released simultaneously to improve the efficiency of the preparation of the pellets, the fabrication for which is described in these examples.

The above descriptions and examples illustrate particular constructions including the preferred embodiments of the solutions. However, the invention is not limited to the precise constructions described herein, but, rather, all modifications and improvements thereof encompassed within the scope of the invention.

I claim:

1. A process for making an aqueous insoluble cellulosic matrix containing a medicament to be utilized in the preparation of orally administered medicaments comprising the steps of:
(I) making an aqueous solution of a cellulosic composition
(II) adding a medicament to the aqueous soluble cellulosic composition and mixing the two to homogeneity.
(III) adding such cellulosic and medicament mixture drop-wise into an aqueous solution of a polyvalent cation metal salt that will react with the cellulosic molecule and result in pellets of an aqueous insoluble cellulosic matrix entrapping the medicament therein, which pellets may then be dried.

2. The process of claim 1 wherein said aqueous-soluble cellulosic agent is selected from a group consisting of ammonium, magnesium, potassium, and sodium salts of alginate or mixtures thereof.

3. The process of claim 1 wherein said aqueous-soluble cellulosic molecule is selected from a group of calcium-sensitive pectins having a degree of esterification (DE) of less than 50% and a degree of amidation (DA) less than 25%.

4. The process of claim 3 wherein non-pectin polysaccharides are added to the pectin composition.

5. The process of claim 4 wherein the non-pectin polysaccharide is selected from the group consisting of carboxy methyl cellulose, carboxy methyl hydroxy ethyl cellulose, hyaluronic acid, carrageenan, alginic acid, sodium alginate, and gellan gum.

6. The process of claim 1 wherein the polyvalent cation is selected from a metal ion derived from salts selected from the group consisting of alkaline earth metal salts, alkali metal salts, transition metal salts, and mixtures thereof.

7. The process of claim 3, wherein the calcium-sensitive pectin is derived from citrus pectin.

8. The process of claim 7, wherein the citrus pectin is selected from the group consisting of lime, lemon, grapefruit, and orange.

9. The process of claim 1 wherein said cation metal salt is selected from the group consisting of calcium, barium, copper, magnesium, iron (ferric or ferrous), zinc, aluminum, manganese, silver, strontium, and mixtures thereof.

10. The process of claim 1 wherein said medicament is a vaccine.

11. The process of claim 1 wherein said medicament is a hormone.

12. The process of claim 1 wherein said medicament is an enzyme.

13. The process of claim 1 wherein said medicament is selected from the group consisting of collagen, maltodextrin, antibiotics, antibacterial agents, anti-inflammatory agents, ascorbic acid, amino acids, antigens and mixtures thereof.

14. The process of claim 1 wherein a plasticizer is added to the cellulosic-medicament composition.

15. The process of claim 14 wherein said plasticizer is selected from a group consisting of glycerin, propylene glycol, ethylene glycol, and polyethylene glycol or mixtures thereof.

16. The process of claim 1 wherein a surface-active agent is added to the cellulosic-medicament composition.

17. The process of claim 16 wherein said surface active agent is selected from a group consisting of polyoxyethylene sorbitan monolaurate, polyoxyethylene sorbitan monopalmitate, polyoxyethylene sorbitan monooleate, polyoxyethylene sorbitan trioleate, polyoxyethylene-polyoxypropylene block polymer, or a mixture thereof.

18. The process of claim 1 wherein the polyvalent cation metal salt complexing the aqueous soluble cellulosic molecule is calcium sulphate.

19. The process of claim 1 wherein the polyvalent cation metal salt complexing the aqueous soluble cellulosic molecule is calcium chloride.

20. A process for making an aqueous insoluble cellulosic matrix containing a medicament to be utilized in the preparation of orally administered medicaments comprising the steps of:
(I) making an aqueous solution of a cellulosic composition.
(II) adding a medicament to the aqueous soluble cellulosic composition and mixing the two to homogeneity.
(III) introducing into the cellulosic-medicament mixture the compound sodium tetraborate,
(IV) adding such cellulosic and medicament mixture drop-wise into an aqueous solution of a polyvalent cation metal salt that will react with the cellulosic molecule and result in pellets of an aqueous insoluble cellulosic matrix entrapping the medicament therein, which pellets may then be dried.

21. The process of claim 20 wherein said aqueous-soluble cellulosic agent is selected from a group consisting of ammonium, magnesium, potassium, and sodium salts of alginate or mixtures thereof.

22. The process of claim 20 wherein said aqueous-soluble cellulosic molecule is selected from a group of calcium-sensitive pectins having a degree of esterification (DE) of less than 50% and a degree of amidation (DA) less than 25%.

23. The process of claim 20 wherein said cation metal salt is selected from the group consisting of calcium, barium, copper, magnesium, iron (ferric or ferrous), zinc, aluminum, manganese, silver, strontium, and mixtures thereof.

24. The process of claim 22 wherein non-pectin polysaccharides are added to the pectin composition.

25. The process of claim 24 wherein the non-pectin polysaccharide is selected from the group consisting of carboxy methyl cellulose, carboxy methyl hydroxy ethyl cellulose, hyaluronic acid, carrageenan, alginic acid, sodium alginate, and gellan gum.

26. The process of claim 20 wherein the polyvalent cation is selected from a metal ion derived from salts selected from the group consisting of alkaline earth metal salts, alkali metal salts, transition metal salts, and mixtures thereof.

27. The process of claim 22, wherein the calcium-sensitive pectin is derived from citrus pectin.

28. The process of claim 27, wherein the citrus pectin is selected from the group consisting of lime, lemon, grapefruit, and orange.

29. The process of claim 20 wherein said medicament is a vaccine.

30. The process of claim 20 wherein said medicament is a hormone.

31. The process of claim 20 wherein said medicament is an enzyme.

32. The process of claim 20 wherein said medicament is selected from the group consisting of collagen, maltodextrin, antibiotics, antibacterial agents, anti-inflammatory agents, ascorbic acid, amino acids, antigens, and mixtures thereof.

33. The process of claim 20 wherein a plasticizer is added to the cellulosic-medicament composition.

34. The process of claim 33 wherein said plasticizer is selected from a group consisting of glycerin, propylene glycol, ethylene glycol, and polyethylene glycol or mixtures thereof.

35. The process of claim 20 wherein a surface-active agent is added to the cellulosic-medicament composition.

36. The process of claim 35 wherein said surface active agent is selected from a group consisting of polyoxyethylene sorbitan monolaurate, polyoxyethylene sorbitan monopalmitate, polyoxyethylene sorbitan monooleate, polyoxyethylene sorbitan trioleate, polyoxyethylene-polyoxypropylene block polymer, or a mixture thereof.

37. The process of claim 20 wherein the polyvalent cation metal salt complexing the aqueous soluble cellulosic molecule is calcium sulphate.

38. The process of claim 20 wherein the polyvalent cation metal salt complexing the aqueous soluble cellulosic molecule is calcium chloride.

39. A process for making an aqueous insoluble cellulosic matrix containing a suspension of cells comprising the steps of:
(I) making an aqueous solution of a cellulosic composition
(II) adding a cellular suspension to the aqueous soluble cellulosic composition and mixing the two to homogeneity
(III) adding such cellulosic and cellular mixture drop-wise into an aqueous solution of a polyvalent cation metal salt that will react with the cellulosic molecule and result in pellets of an aqueous insoluble cellulosic matrix entrapping the cells therein, which pellets may then be dried.

40. The process of claim 39 in which the cells are human tissue cells.

41. The process of claim 39 in which the cells are animal cells.

42. The process of claim 39 in which the cells are microbial cells.

43. The process of claim 39 in which the cells are red blood cells.

44. The process of claim 39 in which the cells are isolated from the Isles of Langerhans of the pancreas and produce insulin.

45. The process of claim 39 in which the cells are cells derived from the blood tissue of humans

46. The process of claim 39 in which the cells are cells derived from the blood tissue of animals.

47. The process of claim 39 in which the cells are plant cells.

48. The process of claim 39 wherein said aqueous-soluble cellulosic agent is selected from a group consisting of ammonium, magnesium, potassium, and sodium salts of alginate or mixtures thereof.

49. The process of claim 39 wherein said aqueous-soluble cellulosic molecule is selected from a group of calcium-sensitive pectins having a degree of esterification (DE) of less than 50% and a degree of amidation (DA) less than 25%.

50. The process of claim 39 wherein said cation metal salt is selected from the group consisting of calcium, barium, copper, magnesium, iron (ferric or ferrous), zinc, aluminum, manganese, silver, strontium, and mixtures thereof.

51. The process of claim 49 wherein non-pectin polysaccharides are added to the pectin composition.

52. The process of claim 51 wherein the non-pectin polysaccharide is selected from the group consisting of carboxy methyl cellulose, carboxy methyl hydroxy ethyl cellulose, hyaluronic acid, carrageenan, alginic acid, sodium alginate, and gellan gum.

53. The process of claim 39 wherein the polyvalent cation is selected from a metal ion derived from salts selected from the group consisting of alkaline earth metal salts, alkali metal salts, transition metal salts, and mixtures thereof.

54. The process of claim 49, wherein the calcium-sensitive pectin is derived from citrus pectin.

55. The process of claim 54, wherein the citrus pectin is selected from the group consisting of lime, lemon, grapefruit, and orange.

56. The process of claim 39 wherein a plasticizer is added to the cellulosic-cell composition.

57. The process of claim 56 wherein said plasticizer is selected from a group consisting of glycerin, propylene glycol, ethylene glycol, and polyethylene glycol or mixtures thereof.

58. The process of claim 39 wherein a surface-active agent is added to the cellulosic-medicament composition.

59. The process of claim 58 wherein said surface active agent is selected from a group consisting of polyoxyethylene sorbitan monolaurate, polyoxyethylene sorbitan monopalmitate, polyoxyethylene sorbitan monooleate, polyoxyethylene sorbitan trioleate, polyoxyethylene-polyoxypropylene block polymer, or a mixture thereof.

60. The process of claim 39 wherein the polyvalent cation metal salt complexing the aqueous soluble cellulosic molecule is calcium sulphate.

61. The process of claim 39 wherein the polyvalent cation metal salt complexing the aqueous soluble cellulosic molecule is calcium chloride.

62. A cellulosic composition containing a medicament which cellulosic composition is made insoluble by being added drop-wise into and thereby being cross-linked with a polyvalent cation metal salt and wherein the aqueous insoluble cross-linked cellulosic pellets contain therein the entrapped and dispersed medicament, which pellets may then be dried.

63. The composition of claim 62 wherein said the cellulosic agent is selected from a group consisting of ammonium, magnesium, potassium, and sodium salts of alginate or mixtures thereof.

64. The composition of claim 62 wherein said the cellulosic molecule is selected from a group of calcium-sensitive pectins having a degree of esterification (DE) of less than 50% and a degree of amidation (DA) less than 25%.

65. The composition of claim 64 wherein non-pectin polysaccharides are added to the pectin composition.

66. The composition of claim 65 wherein the non-pectin polysaccharides are selected from the group consisting of carboxy methyl cellulose, carboxy methyl hydroxy ethyl cellulose, hyaluronic acid, carrageenan, alginic acid, sodium alginate, and gellan gum.

67. The composition of claim 62 wherein the polyvalent cation is selected from a metal ion derived from salts selected from the group consisting of alkaline earth metal salts, alkali metal salts, transition metal salts, and mixtures thereof.

68. The composition of claim 64 wherein the calcium-sensitive pectin is derived from citrus pectin.

69. The composition of claim 68, wherein the citrus pectin is selected from the group consisting of lime, lemon, grapefruit, and orange.

70. The composition of claim 62 wherein the cation metal salt is selected from the group consisting of calcium, barium, copper, magnesium, iron (ferric or ferrous), zinc, aluminum, manganese, silver, strontium, and mixtures thereof.

71. The composition of claim 62 wherein said medicament is a vaccine.

72. The composition of claim 62 wherein said medicament is a hormone.

73. The composition of claim 62 wherein said medicament is an enzyme.

74. The composition of claim 62 wherein said medicament is selected from the group consisting of collagen, maltodextrin, antibiotics, antibacterial agents, anti-inflammatory agents, ascorbic acid, amino acids, antigens and mixtures thereof.

75. The composition of claim 62 wherein a plasticizer is added to the cellulosic-medicament composition.

76. The composition of claim 75 wherein said plasticizer is selected from a group consisting of glycerin, propylene glycol, ethylene glycol, and polyethylene glycol or mixtures thereof.

77. The composition of claim 62 wherein a surface-active agent is added to the cellulosic-medicament composition.

78. The composition 77 wherein said surface active agent is selected from a group consisting of polyoxyethylene sorbitan monolaurate, polyoxyethylene sorbitan monopalmitate, polyoxyethylene sorbitan monooleate, polyoxyethylene sorbitan trioleate, polyoxyethylene-polyoxypropylene block polymer, or a mixture thereof.

79. The composition of claim 62 wherein a polyvalent cation metal salt complexing the cellulosic molecule is calcium sulphate.

80. The composition of claim 62 wherein the polyvalent cation metal salt complexing the cellulosic molecule is calcium chloride.

81. A cellulosic composition containing a medicament and sodium tetraborate, which cellulosic composition is made insoluble by cross-linking with a polyvalent cation metal salt and wherein the aqueous insoluble cross-linked cellulosic composition contains therein the entrapped and dispersed medicament.

82. The composition of claim 81 wherein said the cellulosic agent is selected from a group consisting of ammonium, magnesium, potassium, and sodium salts of alginate or mixtures thereof.

83. The composition of claim 81 wherein said the cellulosic molecule is selected from a group of calcium-sensitive pectins having a degree of esterification (DE) of less than 50% and a degree of amidation (DA) less than 25%.

84. The composition of claim 83 wherein non-pectin polysaccharides are added to the pectin composition.

85. The composition of claim 84 wherein the non-pectin polysaccharides are selected from the group consisting of carboxy methyl cellulose, carboxy methyl hydroxy ethyl cellulose, hyaluronic acid, carrageenan, alginic acid, sodium alginate, and gellan gum.

86. The composition of claim 81 wherein the polyvalent cation is selected from a metal ion derived from salts selected from the group consisting of alkaline earth metal salts, alkali metal salts, transition metal salts, and mixtures thereof.

87. The composition of claim 83 wherein the calcium-sensitive pectin is derived from citrus pectin.

88. The composition of claim 87, wherein the citrus pectin is selected from the group consisting of lime, lemon, grapefruit, and orange.

89. The composition of claim 81 wherein the cation metal salt is selected from the group consisting of calcium, barium, copper, magnesium, iron (ferric or ferrous), zinc, aluminum, manganese, silver, strontium, and mixtures thereof.

90. The composition of claim 81 wherein said medicament is a vaccine.

91. The composition of claim 81 wherein said medicament is a hormone.

92. The composition of claim 81 wherein said medicament is an enzyme.

93. The composition of claim 81 wherein said medicament is selected from the group consisting of collagen, maltodextrin, antibiotics, antibacterial agents, anti-inflammatory agents, ascorbic acid, amino acids, antigens and mixtures thereof.

94. The composition of claim 81 wherein a plasticizer is added to the cellulosic-medicament composition.

95. The composition of claim 94 wherein said plasticizer is selected from a group consisting of glycerin, propylene glycol, ethylene glycol, and polyethylene glycol or mixtures thereof.

96. The composition of claim 81 wherein a surface-active agent is added to the cellulosic-medicament composition.

97. The composition claim 96 wherein said surface active agent is selected from a group consisting of polyoxyethylene sorbitan monolaurate, polyoxyethylene sorbitan monopalmitate, polyoxyethylene sorbitan monooleate, polyoxyethylene sorbitan trioleate, polyoxyethylene-polyoxypropylene block polymer, or a mixture thereof.

98. The composition of claim 81 wherein a polyvalent cation metal salt complexing the cellulosic molecule is calcium sulphate.

99. The composition of claim 81 wherein the polyvalent cation metal salt complexing the cellulosic molecule is calcium chloride.

100. A cellulosic composition containing a cell suspension which cellulosic composition is made insoluble by cross-linking with a polyvalent cation metal salt and wherein the aqueous insoluble cross-linked cellulosic composition contains therein the entrapped and dispersed cells.

101. The composition of claim 100 wherein said the cellulosic agent is selected from a group consisting of ammonium, magnesium, potassium, and sodium salts of alginate or mixtures thereof.

102. The composition of claim 100 wherein said the cellulosic molecule is selected from a group of calcium-sensitive pectins having a degree of esterification (DE) of less than 50% and a degree of amidation (DA) less than 25%.

103. The composition of claim 102 wherein non-pectin polysaccharides are added to the pectin composition.

104. The composition of claim 103 wherein the non-pectin polysaccharides are selected from the group consisting of carboxy methyl cellulose, carboxy methyl hydroxy ethyl cellulose, hyaluronic acid, carrageenan, alginic acid, sodium alginate, and gellan gum.

105. The composition of claim 100 wherein the polyvalent cation is selected from a metal ion derived from salts selected from the group consisting of alkaline earth metal salts, alkali metal salts, transition metal salts, and mixtures thereof.

106. The composition of claim 102 wherein the calcium-sensitive pectin is derived from citrus pectin.

107. The composition of claim 106, wherein the citrus pectin is selected from the group consisting of lime, lemon, grapefruit, and orange.

108. The composition of claim 100 wherein the cation metal salt is selected from the group consisting of calcium, barium, copper, magnesium, iron (ferric or ferrous), zinc, aluminum, manganese, silver, strontium, and mixtures thereof.

109. The composition of claim 100 in which the cells are human tissue cells.

110. The composition of claim 100 in which the cells are animal cells.

111. The composition of claim 100 in which the cells are microbial cells.

112. The composition of claim 100 in which the cells are red blood cells.

113. The composition of claim 100 in which the cells are isolated from the Isles of Langerhans of the pancreas and produce insulin.

114. The composition of claim 100 in which the cells are cells derived from the blood tissue of humans.

115. The composition of claim 100 in which the cells are cells derived from the blood tissue of animals.

116. The composition of claim 100 in which the cells are plant cells.

117. The composition of claim 100 wherein a plasticizer is added to the cellulosic-cell composition.

118. The composition of claim 117 wherein said plasticizer is selected from a group consisting of glycerin, propylene glycol, ethylene glycol, and polyethylene glycol or mixtures thereof.

119. The composition of claim 100 wherein a surface-active agent is added to the cellulosic-cell composition.

120. The composition 119 wherein said surface active agent is selected from a group consisting of polyoxyethylene sorbitan monolaurate, polyoxyethylene sorbitan monopalmitate, polyoxyethylene sorbitan monooleate, polyoxyethylene sorbitan trioleate, polyoxyethylene-polyoxypropylene block polymer, or a mixture thereof.

121. The composition of claim 100 wherein a polyvalent cation metal salt complexing the cellulosic molecule is calcium sulphate.

122. The composition of claim 100 wherein the polyvalent cation metal salt complexing the cellulosic molecule is calcium chloride.
